(19) United States
(12) Patent Application Publication
(10) Pub. No.: US 2023/0002807 A1
(43) Pub. Date: Jan. 5, 2023

(54) METHODS AND COMPOSITIONS FOR NUCLEIC ACID ANALYSIS

(71) Applicant: FLUENT BIOSCIENCES INC., Watertown, MA (US)

(72) Inventors: Tony Godfrey, Watertown, MA (US); Catherine Kugler, Watertown, MA (US); Robert Meltzer, Belmont, MA (US)

(21) Appl. No.: 17/853,023

(22) Filed: Jun. 29, 2022

Related U.S. Application Data
(60) Provisional application No. 63/216,961, filed on Jun. 30, 2021.

(51) Int. Cl. C12Q 1/6806; C12N 15/10

(52) U.S. Cl. CPC C12Q 1/6806; C12N 15/1068

(57) ABSTRACT

This invention provides ultra-sensitive methods and compositions for detecting patient-specific mutations from cell free nucleic acids (cfDNA) without sequencing. Methods of the invention make use of fluidic partitions for multiplex amplification of cfDNA and thereby create a library of uniformly amplified amplicons. The uniformly amplified amplicons can be split into any number of different detection reactions (while maintaining detection sensitivity) for single-plex detection of mutations present in cfDNA. These methods provide substantially improved signal to noise ratio and easier discrimination of low-abundance mutations.

### TECHNICAL FIELD

This invention provides methods and compositions for analyzing cell-free DNA.

### BACKGROUND

Cell-free DNA (cfDNA) is released by cancer cells as a result of necrosis. Since cfDNA is present in body fluid, e.g., blood or urine, and can inform on disease status, methods of analyzing cfDNA are of great clinical interest. To gain useful insights from cfDNA, specific regions of the cfDNA must be detected and analyzed to identify mutations absent from normal, wide-type cells. The mutations are generally detected using DNA sequencing technologies. Unfortunately, despite rapid advancements in DNA sequencing, several challenges remain to identifying cancer mutations in cfDNA.

For example, mutant cfDNA from cancer cells is extremely rare compared to the background of normal DNA. Accordingly, ultra-sensitive detection modalities, e.g., below 0.1%, are required. Unfortunately, standard sequencing technologies are limited by 0.5 to 1.0% base calling error and as such, sophisticated library preparation methods and bioinformatic analysis are required for mutation detection. This makes preparing cfDNA sequencing libraries complicated, time-consuming and expensive. Moreover, since cfDNA must be extracted from body fluid, the cfDNA is typically degraded to very short fragment lengths, which are often below the detection limit required for DNA sequencing.

Accordingly, despite wide-spread recognition of the potential for cfDNA applications in early cancer detection and/or monitoring, successful strategies to gain clinically relevant information from cfDNA remain lacking.

### SUMMARY

This invention provides ultra-sensitive methods and compositions for detecting mutations from cell free DNA (cfDNA). The invention makes use of pre-templated instant partitions for multiplex pre-amplification of diagnostic targets before those targets are assessed for cancer mutations. Since the pre-templated instant partitions provide highly uniform multiplex amplification of target cfDNA, methods of the invention are useful to substantially increase the total number of diagnostic targets and technical replicates used for mutation detection. Accordingly, the amplified targets can be divided across any number of detection reactions for the detection of specific cancer mutations. This allows patient-specific cancer mutations to be detected by ultra-sensitive, PCR-based modalities that are generally limited in their multiplexing capacities. As such, this invention provides a robust and inexpensive workflow for detecting patient-specific cancer mutations in a rapid sample-to-answer format without expensive sequencing.

Specifically, methods and compositions of the invention make use of pre-templated instant partitions to produce uniformly sized reaction chambers from which targets of cfDNA are amplified. Preferably, a plurality of targets across one or more different cfDNA molecules are amplified, thereby increasing the total number of distinct targets by multiplex amplification for cancer detection. Multiplexed targets of cfDNA are uniformly enriched inside partitions without amplification bias. The enriched target amplicons can be split into any number of different reaction vessels for mutation detection. This allows for multiple, highly sensitive single-plex detection reactions to be performed in parallel on standard laboratory equipment, e.g., dPCR instrumentation, without any concern of detection sensitivity reduction due to sample splitting, which is a common problem of current technologies.

Accordingly, this invention provides a simplified, cost-effective workflow with the potential to transform personalized medicine for detecting disease, monitoring recurrence, or assessing the efficacy of disease therapy.

The invention provides methods and compositions involving multiplex amplification of targets of cfDNA with pre-templated instant partitions. The targets may be a single base mutation present at an extremely low frequency (less than 0.01%) within a body fluid sample, e.g., blood, urine, or sputum. The target may be a patient—specific mutation indicative of disease recurrence. Following target amplification, methods of the invention include ultra-sensitive detection strategies based on PCR (e.g., qPCR), which are fast, reliable, and cost effective. As such, compositions and methods of the invention are particularly well suited for tracking disease and/or monitoring cancer recurrence.

For example, in some embodiments, methods of the invention involve longitudinal monitoring of disease.

Longitudinal monitoring generally involves repeated, intra-patient assessments of cancer mutations over time. Repeat measurements may be used to determine if a patient's disease is stable, to show how a patient is responding to a treatment, or to reveal the effect of a treatment. Since cfDNA can be obtained non-invasively, e.g., from urine or blood, and methods of the invention are useful to rapidly detect rare tumor mutations, the invention is well suited for making repeat measurements over time. Accordingly, compositions and methods of the disclosure provide tools for not only detecting disease, but also for longitudinal patient monitoring of patients undergoing therapy. In particular, the invention allows for the rapid and inexpensive qualification of disease and rapid determination of a subjects' response to therapy.

In one aspect, the invention provides methods for detecting a mutation from a nucleic acid, such as cfDNA. The method involves preparing an aqueous solution of a target nucleic acid and PCR primers inside a reaction vessel, such as a 1.5 milliliter tube. The PCR primers should include sequences complementary to one or more regions of the target nucleic acid. For example, the PCR primers may have sequences complementary to one or more regions of different fragments of cfDNA. An oil is added to the vessel to create a mixture. The method further includes shearing the mixture to form a plurality of water-in-oil partitions, such that at least a portion of the partitions includes a single target nucleic acid and the PCR primers. The method further includes amplifying the target nucleic acid inside the partitions with the PCR primers to produce a library of amplicons. The amplicons are split into a plurality of different reaction vessels. Preferably, one or more of the reaction vessels contains reagents (e.g., primers) useful for detecting the presence of a mutation by PCR. For example, the vessels may contain primers with sequences sensitive to a cancer mutation. The method involves detecting and/or quantifying the amplicons by PCR to assess disease. In preferred embodiments, to avoid expensive and time-consuming detection of cancer mutations, the detecting of amplicons is performed by quantitative PCR (qPCR). Preferably, detection is performed with primers that are sensitive to cancer mutations.

To facilitate quantitative analysis, the invention includes experimental conditions that ensure uniform pre-amplification of cfDNA targets. Methods of the invention include adding PCR primer pairs at concentrations that are pre-calculated to guarantee substantially all primers for an amplifiable species within a reaction (inside a droplet containing nucleic acid complementary to the primers) are consumed within a specified number of PCR amplification cycles. This is useful to safeguard uniform amplification of cfDNA targets despite unequal amplification efficiencies of some targets. For example, in a multiplexed PCR reaction, some targets of cfDNA may amplify efficiently, whereas some targets of cfDNA may amplify inefficiently. In bulk amplification, the efficient targets of cfDNA may outcompete the inefficient species, leading to a wide variation in amplification uniformity between efficient and inefficient species, which precludes accurate quantification.

Methods of the invention can ensure uniform amplification of cfDNA by at least two independent mechanisms. First, when the sample of cfDNA is partitioned, there is on average one or zero amplifiable fragments present in each partition. This provides reduced opportunity for competition between amplified products (amplicons). Next, in preferred embodiments, target specific PCR primers are limited in concentration, such that substantially all the PCR primers are consumed during PCR inside those partitions that have cfDNA complementary to the primers. The PCR amplification protocol is specified such that all primers are consumed even for inefficient targets. Since the pre-templated instant partitions produce partitions of uniform partition volumes, the resulting concentration of amplicons in each partition can therefore be normalized. Thus, in some embodiments, conditions are established to ensure efficient and uniform amplification. In one aspect, a sample is partitioned such that on average less than one amplifiable target is present in each partition, thus reducing competition between amplified products in the partition. In addition, in one aspect the invention provides for limiting concentrations of target-specific primers such that the primers are substantially consumed over repeated PCR cycles. In the case of uniform partition volumes, the resulting concentration of amplicons is normalized. This alleviates the concern that in some multiplex PCR reactions some targets are amplified efficiently and some relatively inefficiently. The efficient amplifications are likely to outcompete the inefficient amplifications, leading to non-uniform populations of amplicons.

Accordingly, in some embodiments of the invention a number of PCR primers consumed inside a significant number of individual partitions that include complementary cfDNA is less than a number of PCR cycles performed during the amplification. Alternatively, or additionally, a number of unused PCR primers inside those individual partitions reaches zero before a final PCR cycle is performed. Accordingly, amplifying cfDNA targets including pre-templated instant partitions can involve at least one PCR cycle having substantially zero amplification events. As such, in preferred methods of the invention the target nucleic acid is uniformly amplified during the amplification step.

Methods of the invention use pre-templated instant partitions for precise amplification of cfDNA targets. The pre-templated instant partitions are generally formed using template particles that template the formation of the partitions inside the vessel, thereby creating partitions encapsulating cfDNA inside a defined volume of fluid, and thus defined reagent concentrations. Accordingly, in some embodiments, before shearing the mixture, the method further includes adding template particles to the aqueous solution. The template particles template the formation of substantially uniform partitions comprising PCR primers in substantially uniform concentrations.

Cancer-derived cfDNA can be identified from urine. Unfortunately, the cfDNA is generally highly fragmented, which limits targeted analysis of genomic alterations by conventional analytic methods, such as sequencing. Surprisingly, however, there is a consistent distribution of fragment sizes of cfDNA with a modal size of 80 to 81 base pairs, suggesting non-random fragmentation. This non-random fragmentation pattern may be the result of chromatin accessibility and/or gene expression from the contributing cancer cells.

Accordingly, in some embodiments of the invention, the target nucleic acid is approximately 60-90 base pairs in length, for example, approximately 80 or 81 base pairs in length. The PCR primers can be designed to amplify regions of cfDNA that correlate with recurrently protected genomic regions. The recurrently protected genomic regions can involve sequences of DNA that are highly expressed in cancer cells or regions associated with high chromatin accessibility. In some embodiments, methods of the invention may include comparing cancer sequence information obtained by whole genome DNA (e.g., cfDNA) sequencing of a subject with a cancer to corresponding wild-type sequences obtained by whole genome DNA sequencing of a subject without cancer to identify recurrently protected genomic regions associated with a certain cancer. Accordingly, PCR primers can be designed that are complementary to recurrently protected genomic regions, e.g., the top 5% or 1% of recurrently protected genomic regions.

Methods of the invention allow for the detection of patient-specific mutations without sequencing. As such, methods of the invention provide cost-effective workflows for monitoring disease. For example, preferred embodiments involve detecting target amplicons by qPCR. Detection may be performed with primers that are sensitive to mutations. For example, in some instances, qPCR primers comprise sequences that prevent hybridization to target amplicons having a mutated sequence. Accordingly, target amplicons comprising a mutated sequence may fail to amplify. Failure of one or more target amplicons to amplify during qPCR can be detected according to embodiments of the invention and quantified to assess a health status of a patient. In other instances, qPCR primers are designed with sequences that are highly specific towards target nucleic acids comprising a certain mutation, such as a single base pair substitution.

In some embodiments, qPCR is performed with modified primers. The modified primers may comprise modifications that enhance primer specificity for one or more tumor mutations. For example, the modified primers may include one of locked nucleic acid primers, 2-tail primers, or fluorogenic primers. In some instances, the primers are provided as part of a kit that includes template particles, and reagents for performing multiplex amplification inside pre-templated instant partitions before qPCR.

### BRIEF DESCRIPTION OF THE DRAWINGS

FIG. 1 shows a method of detecting a mutation.

FIG. 2 diagrams a method for detecting rare nucleic acids.

FIG. 3 shows a fluorescent image of dPCR-amplified cfDNA inside partitions.

FIG. 4 shows data from a pre-amplification assay.

FIG. 5 shows qPCR data comparing amplification efficiencies of pre-amplified targets vs non-amplified targets.

FIG. 6 provides qPCR data that demonstrate the ability of methods of the invention to discriminate between mutant and wild-type nucleic acid.

### DETAILED DESCRIPTION

The invention provides methods useful for detecting ultra-rare mutations from cell free DNA (cfDNA) in high throughput without sequencing. Accordingly, this invention provides simple, inexpensive, and rapid sample-to-result workflows useful for detecting disease, monitoring disease recurrence, or assessing the efficacy of a therapy.

The invention provides methods for the use of pre-templated instant partitions for patient-specific detection of rare mutations from a background of normal DNA, which is generally expected in cfDNA preparations from body fluid. In preferred embodiments, the invention combines uniform, high-fidelity multiplexed target pre-amplification (i.e., multiplex amplification of targets before detection) of cfDNA inside the pre-templated instant partitions with qPCR-based, single-plex detection. The pre-templated instant partitions make use of hydrogel template particles that template the formation of, for example, thousands to millions of partitions simultaneously, in a single tube, and segregate fragments of cfDNA inside those partitions for amplification prior to qPCR analysis.

The pre-templated instant partitions allow for the uniform pre-amplification of target cfDNA with precise fluidic control, and as such, overcome current PCR limitations for an inexpensive approach to detecting patient-specific mutations. Since the pre-templated partitions can be formed around templated particles of a consistent shape and size, the resultant partitions containing one or zero fragments of cfDNA also include a well-defined sample volume. The number of partitions is defined only by the reaction vessel size and the quantities of reagents, e.g., template particles. Accordingly, methods of the invention are also massively scalable.

By isolating individual, amplifiable cfDNA fragments inside partitions, the invention can eliminate or substantially reduce amplification bias, and thereby allow for the amplification trace amounts (e.g., less than 0.01%) of a target nucleic acid. For example, amplification of cfDNA inside individual partitions, in parallel, can eliminate unwanted amplicon-to-amplicon interactions and thus permit uniform amplification across multiplexed targets.

Uniform target cfDNA amplification allows the resulting mixture of enriched amplicons to be split into any number of reaction vessels (e.g., PIPs) for detection, e.g., 1, 2, 3, 4, 5, 10, 20, 30, 40, 50, 60, 70, 80, 90, 100, 1000, or more, different reaction vessels. The reaction vessels may comprise individual wells of a multi-well dish.

Preferably, a substantial number of the wells contain reagents for detecting at least one cancer mutation. For example, one or more wells may contain a reagent (e.g., a primer) for detecting a first cancer mutation, while different ones of the wells may contain a different reagent for detecting a different cancer mutation. This allows for multiple single-plex detection reactions to be performed in parallel on standard laboratory equipment, such as a qPCR instrumentation, and eliminates the concern of detection sensitivity reduction due to sample splitting common in competing technologies.

By combining pre-templated instant partitioning with PCR-based detection modalities, the invention is useful to uniformly enrich selected regions of cfDNA that are of clinical interest and enable sample-splitting for ultra-sensitive mutation detection without any loss of sensitivity. Furthermore, some embodiments involve digital pre-amplification. Digital pre-amplification can increase the absolute target copy number (even after sample splitting) and overcome certain sensitivity issues associated with some PCR-based detection strategies of otherwise low-abundance (<10 copies) variants. Finally, enriching the targets of interest with pre-amplification (amplification events that take place inside partitions) will suppress non-target genomic background resulting in improved specificity.

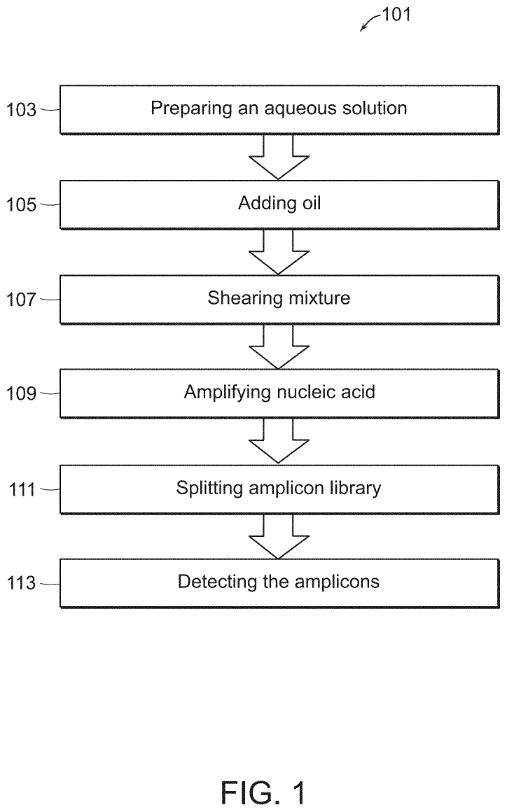

FIG. 1 shows a method 101 of detecting a mutation. The method 101 involves preparing 103 an aqueous solution comprising a target nucleic acid and PCR primers. The PCR primers include sequences that are complementary to the target nucleic acid. The method 101 further includes combining 105 the aqueous solution with an oil to create a mixture. Next, the mixture is sheared 107 the mixture into a plurality of water-in-oil partitions, wherein at least a portion of the water-in-oil partitions includes a single target nucleic acid and PCR primers. After shearing 107, the method involves amplifying 109 target nucleic acids inside the partitions with the PCR primers to produce a library of amplicons. The method further includes splitting 111 the library of amplicons by depositing a portion of amplicons into a plurality of different reaction vessels. Next, the method involves detecting 113 and/or quantifying the amplicons from the plurality of reaction vessels to thereby analyze the nucleic acid.

More particularly, the first step of the method 101 involves preparing 103 an aqueous solution. While any suitable order may be used to prepare 103 the aqueous solution, it may be useful to first provide a tube that includes template particles. The template particles may be provided in an aqueous media (e.g., saline, nutrient broth, water) or dried to be rehydrated at time of use. A sample containing the target nucleic acid (e.g., cfDNA) may be added into the tube, for example, directly upon sample collection from a subject, or after some minimal sample prep step such as ethanol purification. Preferably, the sample is a body fluid sample. For example, a blood sample or a urine sample. Accordingly, the sample can be collected from the subject by non-invasive methods, such as blood draw or urine collection.

The target nucleic acid is preferably cfDNA. cfDNA are generally degraded DNA fragments (50-200 bp) released to the blood plasma. cfDNA includes various forms of DNA freely circulating in the bloodstream, including circulating tumor DNA (ctDNA), cell-free mitochondrial DNA (cf mtDNA), and cell-free fetal DNA (cffDNA).

Elevated levels of cfDNA are observed in cancer, especially in its advanced states. There is evidence that cfDNA becomes increasingly frequent in circulation with the onset of age. cfDNA has been shown to be a useful biomarker for a multitude of ailments other than cancer and fetal medicine. This includes but is not limited to trauma, sepsis, aseptic inflammation, myocardial infarction, stroke, transplantation, diabetes, and sickle cell disease. Accordingly, methods of the invention are useful for detection of not just cancer, but also trauma, sepsis, aseptic inflammation, myocardial infarction, stroke, transplantation, diabetes, and sickle cell disease. cfDNA is mostly a double-stranded extracellular molecule of DNA, consisting of small fragments (50 to 200 bp) and larger fragments (21 kb) and has been recognized as an accurate marker for the diagnosis of prostate cancer and breast cancer.

The release of cfDNA into the bloodstream appears by different mechanisms s, including the primary tumor, tumor cells that circulate in peripheral blood, metastatic deposits present at distant sites, and normal cell types, like hematopoietic and stromal cells. Tumor cells and cfDNA circulate in the bloodstream of patients with cancer. Its rapidly increased accumulation in blood during tumor development may be caused by an excessive DNA release by apoptotic cells and necrotic cells. cfDNA may circulate predominantly as nucleosomes, which are nuclear complexes of histones and DNA. They are frequently nonspecifically elevated in cancer but may be more specific for monitoring cytotoxic cancer therapy, mainly for the early estimation of therapy efficacy.

Preparing 103 an aqueous solution includes adding DNA amplification reagents, such as, DNA polymerase, deoxynucleotide triphosphates (dNTPs), reaction buffer, magnesium, and PCR primers. The PCR primers are generally synthetic primers that are chemically synthesized oligonucleotides, usually of DNA, which can be customized to anneal to a specific site on the template DNA. One or more of the PCR primers may be fluorogenic. In solution, the primer spontaneously hybridizes with the template through Watson-Crick base pairing before being extended by DNA polymerase. The PCR primers are typically between 18 and 24 bases in length and code for specific upstream and downstream sites of the target nucleic acid sequence being amplified. The primers can be designed and ordered from a third party, such as from the company operating under the trade name Thermo Fisher Scientific. Methods for designing the primers are discussed below.

To facilitate quantitative analysis, the PCR primers are added to the solution at a defined concentration that is pre-calculated to ensure that a substantial number of PCR primers are consumed within a specified number of PCR amplification cycles. In particular, the concentration of each species of PCR primers is added to the aqueous solution at a certain concentration calculated such that, during amplification of complementary, target nucleic acid inside a partition, the PCR primers are consumed. This is useful to ensure uniform amplification of cfDNA targets despite unequal amplification efficiencies of some targets.

For example, in a multiplexed PCR reaction, some targets may amplify efficiently, whereas some targets may amplify inefficiently. In bulk amplification, the efficient targets may outcompete the inefficient species, leading to a wide variation in amplification uniformity between efficient and inefficient species, which may preclude accurate quantification. Accordingly, target specific PCR primers are limited in concentration, such that substantially all the PCR primers are consumed during PCR inside those partitions that have target nucleic acid. The PCR amplification protocol is specified such that all primers are consumed even for inefficient targets. Since the pre-templated instant partitions produce partitions of uniform partition volumes, the resulting concentration of amplicons in each partition can therefore be normalized. Calculating the concentration of PCR primers to add to the aqueous solution can be achieved using simple arithmetic to calculate an approximate number of available primer molecules per pre-templated instant partition. The number of available primer molecules per pre-templated instant partition can be calculated as a function of reaction volume, size of the template particle, sample input, scale of multiplex, and bulk primer concentration.

Accordingly, in some embodiments of the invention a number of PCR primers consumed inside a significant number of individual partitions, e.g., those that include complementary target nucleic acid, is less than a number of PCR cycles performed during the amplification.

Alternatively, or additionally, a number of unused PCR primers inside those individual partitions reaches zero before a final PCR cycle is performed. Accordingly, amplifying target nucleic acid including pre-templated instant partitions can involve at least one PCR cycle having substantially zero amplification events. As such, in preferred methods of the invention the target nucleic acid is uniformly amplified during the amplification step.

The PCR primers include sequences complementary to a target nucleic acid. Preferably the target nucleic acid is cfDNA isolated from urine. cfDNA from urine is a promising analyte for noninvasive diagnostics. However, urine cfDNA is highly fragmented. Whether characteristics of these fragments reflect underlying genomic architecture is unknown. Characterization of cfDNA fragments by whole-genome sequencing has revealed multiple strong peaks between 40 and 120 base pairs (bp) with a modal size of 81- and sharp 10-bp periodicity, suggesting transient protection from complete degradation, as discussed in Markus, 2021, Analysis of recurrently protected genomic regions in cell-free DNA found in urine, Science Translational Medicine, 13(581), incorporated by reference. Accordingly, in some embodiments, the target is pre-identified as a recurrently protected genomic region of DNA. The recurrently protected genomic region may be pre-identified by comparing genome-wide differences of cfDNA sequences taken from a patient inflicted with cancer and comparing those sequences with cfDNA sequences of a normal, healthy patient (i.e., without cancer). Accordingly, in some embodiments of the method 101, the target nucleic acid is approximately 60-90 base pairs in length, for example, approximately 75-85 base pairs in length, and preferably, about 80-81 base pairs in length.

Next, the method 101 involves adding 105 oil to the aqueous solution. The oil is added to the tube (which will typically initially overlay the aqueous mixture). In some embodiments, one or more surfactants, described below, may also be added to the mixture to stabilize partitions. The method 101 then includes shearing 107 the mixture to shear the fluid causing partitioning. It may be found that during the vortexing: the mixture partitions into the aqueous droplets within about 5 to about 50 seconds, e.g., about 30 seconds. Shearing 107 the mixture is preferably performed by vortexing. Vortexing is preferred for its ability to reliably generate partitions of a uniform size distribution. Uniformity of partitions is helpful to ensure each “reaction chamber” is provided with a substantially equal volume and thus substantially equal reagents. Vortexing is also easily controlled (e.g., by controlling time and vortex speed) and thus produces data that are more easily reproducible. Vortexing may be performed with a standard bench-top vortexer or a vortexing device as described in co-owned U.S. patent application Ser. No. 17/146,768, which is incorporated by reference.

Next, the method involves amplifying 109 the target nucleic acid inside the partitions to produce a library of target amplicons. Target amplification is carried out by PCR, which may be performed using a thermocycler.

Preferably, the number of PCR cycles are selected such that—inside those partitions having target nucleic acid—the PCR primers are exhausted prior to the final cycle of DNA amplification. For example, as discussed in Fenrich and Flynn, Preamplification and How it Can Be Used to Maximize qPCR Data Generation from Limited Samples, 2018, BioRad, which is incorporated by reference. This is useful to achieve uniform amplification of the target nucleic acid. Amplifying 109 may involve digital PCR. Accordingly, amplifying 109 may be performed in the presence of a fluorophore. A fluorophore is a fluorescent chemical compound that can re-emit light upon light excitation. The fluorescent dye may be a part of a probe, such as a hydrolysis probe. In certain aspects, the fluorophore is incorporated into an amplicon (e.g., as an intercalating dye), which is made by copying the primer-bound target nucleic acid with a DNA polymerase. The presence of the fluorophore allows the amplicon to be detected. The PCR primers may comprise (e.g., be linked with) the fluorophore.

The library of target amplicons is then split 111 (divided) into different reaction vessels for detection 113 of cancer mutations with PCR-based strategies. For example, the library of amplicons may be diluted (e.g., 100x) in a solution and split 111 by pipetting the solution containing the amplicons into wells (e.g., 6, 12, 24, 48, 96 wells) of a multi-well plate. Preferably, the wells contain reagents for detecting at least one cancer mutation. The wells can be labeled to identify a mutation that a regent corresponding reagent is intended to detect. For example, one well may contain a reagent for detecting a first cancer mutation, while a second well of the multi-well plate may contain a different reagent for detecting a second cancer mutation. The reagents are preferably primers that are sensitive to sequence mutations. This allows for multiple single-plex detection reactions to be performed in parallel on standard laboratory equipment, such as a qPCR instrumentation, and eliminates the concern of detection sensitivity reduction due to sample splitting common in competing technologies. Subsequently, the amplicons are quantified 113 by PCR, e.g., qPCR.

Detection 113 can further involve quantification of detected cancer mutations. For example, the cancer mutations can be quantified by counting a total number of each one of the detected mutations. The total number of each mutation can be used to calculate a frequency of one or more of the different mutations. That is, a total of each mutation can be divided by a total number of one or more different mutations (e.g., the total number of detected mutations) to determine a relative frequency with which one mutation appears relative to another one of the detected mutations. The frequency at which certain mutations appear can provide information that is useful to track cancer progression. For example, the frequencies at which mutations appear can be useful to track tumor clonal evolution.

In some embodiments detection 113 is carried out using quantitative PCR (qPCR) based strategies. qPCR monitors the amplification of a target DNA molecule during the PCR (i.e., in real time), not at its end, as in conventional PCR. qPCR can be used quantitatively and semi-quantitatively. Two common methods for the detection of PCR products by qPCR are (1) non-specific fluorescent dyes that intercalate with any double-stranded DNA and (2) sequence-specific DNA probes consisting of oligonucleotides that are labelled with a fluorescent reporter, which permits detection only after hybridization of the probe with its complementary sequence.

Preferably qPCR is performed using primers that are highly sensitive to mutations in nucleic acid. For example, the primers may be modified primers that are sensitive to a single base pair substitution. Accordingly, in some preferred embodiments, different reaction vessels will contain different primers sensitive to different mutations. For example, in some instances, a primer is designed with a sequence that will not hybridize to nucleic acid inside the reaction vessel if the nucleic acid comprises a mutation, such as a single base pair substitution. In other instances, the primers are designed such that the primers will only hybridize to nucleic acid inside the reaction vessel if the amplicon includes a specific cancer mutation, such as a single base pair mutation.

qPCR is carried out with a thermal cycler that has the capacity to illuminate each sample with a beam of light of at least one specified wavelength and detect fluorescence emitted by an excited fluorophore that is reflective of an amplicon. The thermal cycler is also able to rapidly heat and chill samples, thereby taking advantage of the physicochemical properties of the nucleic acids and DNA polymerase. The qPCR process generally consists of a series of temperature changes that are repeated (e.g., 15-25 times). These cycles normally consist of three stages: the first, at around 95° C., allows the separation of the amplified target nucleic acid's double chain; the second, at a temperature of around 50-60° C., allows the binding of the primers with the DNA template; the third, at between 68— 72° C., facilitates the polymerization carried out by a DNA polymerase. Due to the small size of the fragments the last step may be omitted as the DNA polymerase enzyme may be able to replicate the amplicon during the change between the alignment stage and the denaturing stage. In addition, in four-step PCR the fluorescence is measured during short temperature phases lasting only a few seconds in each cycle, with a temperature of, for example, 80° C., in order to reduce the signal caused by the presence of primer dimers when a non-specific dye is used. The temperatures and the timings used for each cycle depend on a wide variety of parameters, such as: the enzyme used to synthesize the DNA, the concentration of divalent ions and deoxyribonucleotides (dNTPs) in the reaction and the bonding temperature of the primers.

In some embodiments implementing qPCR, a DNA-binding dye binds to all double-stranded (ds) DNA in the PCR reaction, increasing the fluorescence quantum yield of the dye. An increase in DNA product during PCR therefore leads to an increase in fluorescence intensity measured at each cycle. Allowing quantification of the target nucleic acid.

In real-time PCR with dsDNA dyes the reaction is prepared as usual, with the addition of fluorescent dsDNA dye. Then the reaction is run in a real-time PCR instrument, and after each cycle, the intensity of fluorescence is measured with a detector; the dye only fluoresces when bound to the dsDNA (i.e., the PCR product). This method has the advantage of only needing a pair of primers to carry out the amplification, which keeps costs down; multiple target sequences can be monitored in a tube by using different types of dyes.

Some embodiments of the invention involve digital PCR (dPCR) amplification followed by qPCR. dPCR is an amplification reaction in which dilute samples are divided into many separate reactions. See for example, Brown et al. (U.S. Pat. Nos. 6,143,496 and 6,391,559), Vogelstein et al. (U.S. Pat. Nos. 6,440,706, 6,753,147, and 7,824,889), as well as Larson et al (U.S. patent application Ser. No. 13/026,120), Link et al. (U.S. patent application Ser. Nos. 11/803,101, 11/803,104, and 12/087,713), and Anderson et al (U.S. Pat. No. 7,041,481, which reissued as RE41,780), the content of each of which is incorporated by reference herein in its entirety. dPCR has very high sensitivity but is generally limited in its multiplexing ability. Samples may be split into multiple dPCR reactions, but that has conventionally necessitated a reduction in assay sensitivity in samples with stochastically limited targets as in clinical cfDNA samples. Advantageously, methods of the invention address this limitation by the pre-amplification of target nucleic acid inside pre-templated instant partitions.

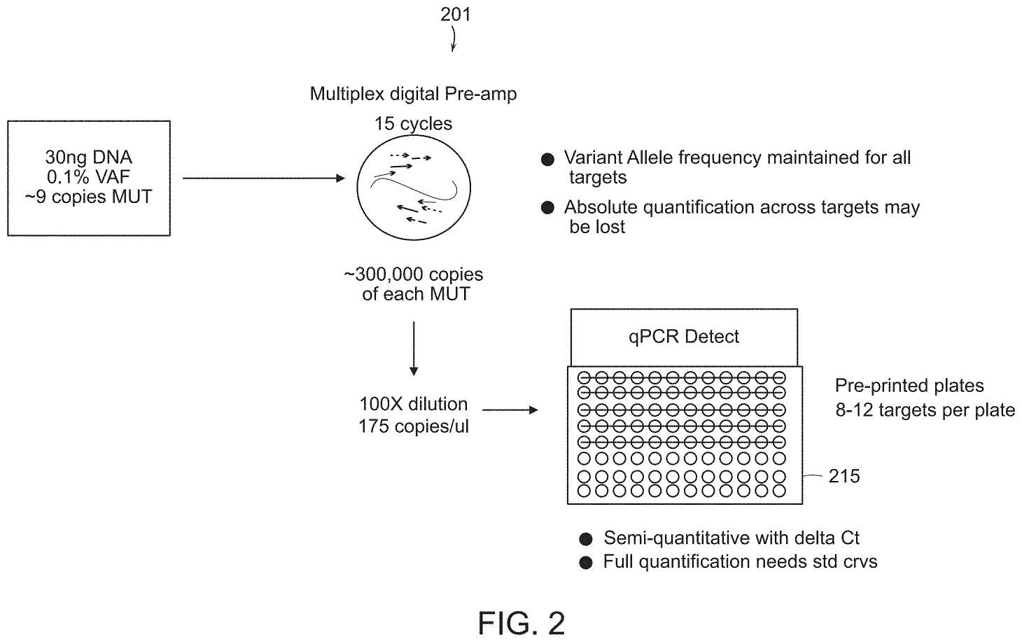

FIG. 2 diagrams a method 201 for detecting rare nucleic acids. The method 201 involves digital pre-amplification of target nucleic acid (cfDNA) followed by qPCR. Digital pre-amplification of cfDNA allows for expansion of available target molecules while maintaining the proportionality of targets (i.e. wild type and mutant representations in a single gene). This allows a sample to be split into multiple single plex qPCR assays, which are relatively simple to design and optimize.

Preferably, the method 201 uses emulsions to segregate low quantities (30 nanograms of DNA total including ˜9 mutated “MUT” fragments) of cfDNA into individual droplets or partitions for dPCR amplification. Individual amplifiable fragments of cfDNA are isolated with a pre-determined mixture of PCR amplification primers in pre-templated instant partitions. The partitions allow for multiplex amplification of target cfDNA by dPCR while enhancing PCR amplification uniformity. For example, the partitions reduce competition of multiple amplified products.

The cfDNA targets are amplified, e.g., by 15 cycles of dPCR. The result is a normalized amplification yield (·300,000 copies of mutated fragments) across targets. This has been demonstrated by the inventors in targeted sequencing applications. dPCR amplification may be performed in shaken emulsions. Preferably, however, amplification is performed in templated emulsions, i.e., emulsions formed using template particles. Amplification uniformity in pre-templated emulsions is preferred because the template particles establish a consistent volume for individual reaction volumes.

Digital pre-amplification of the target cfDNA produces a library of amplification products referred to as amplicons. After digital pre-amplification, the method 201 involves detecting and or quantifying the amplicons by qPCR. This can involve diluting the library of amplicons by 100x, such that approximately 175 copies of each MUT are present per microliter of solution. The diluted solution may be aliquoted into separate reaction vessels of a multi-well plate 215 for single-plex detection. The reaction vessels may be pre-prepared with reagents for detection of specific cancer mutations. Accordingly, one well may contain a reagent (e.g., a primer) useful for detection a first mutation, while a second well of the multi-well plate is prepared with a different reagent for detecting a second mutation, etc. The wells should be labeled to identify the mutation that is detected. qPCR may be quantitative or semi-quantitative.

qPCR may be performed with modified primers. The primers are preferably sensitive to mutations. That is, the primers are preferably designed such that if a target amplicon is mutated (contains a sequence of nucleotides not found in wild-type normal DNA sequence) the primer will not hybridize to the target amplicon, and as such, amplification will not occur. The lack of an amplification event may be indicative of the presence of a mutation.

For example, one or more of the qPCR primers may be a locked nucleic acid (LNA) primer. An LNA primer includes a novel nucleic acid analog that contains a 2′-0, 4′-C methylene bridge. This bridge—locked in the 3′-endo conformation—restricts the flexibility of the ribofuranose ring and locks the structure into a rigid bicyclic formation. This confers enhanced qPCR assay performance. For example, the LNA primer can provide increased thermal stability and hybridization specificity, more accurate gene quantification and allelic discrimination, as well as easier and more flexible designs for problematic target sequences. For example, as described in Ballantyne, 2008, Locked nucleic acids in PCR primers increase sensitivity and performance, Genomics, 91(3):301-5, which is incorporated by reference. In some embodiments, the method may use a 2-tail primer to achieve very high specificity with a very short mutation detection probe (6 to 8 bases). The affinity is enhanced by including a covalently connected but distal primer portion that binds to an additional portion of the target sequence.

In some embodiments, one or more of the primers are optimized for detection of single nucleotide substitutions. For example, one or more of the primers may comprise a SuperSelective primer consisting of a 5′ anchor portion that hybridizes to the target followed by a noncomplementary “bridge” and “foot” portion corresponding to a target allele. The foot sequence may be short, such that a single mismatch at the terminal 3′ nucleotide destabilizes primer binding and prevents extension, enabling discrimination of different alleles. For example, as described in Touroutine and Tanis, 2020, A rapid, SuperSelective method for detection of single nucleotide variants in Caenorhabditis elegans, Genetics, 216(2): 343-352, and also in Vargas, 2016, Multiplex Real-Time PCR Assays that Measure the Abundance of Extremely Rare Mutations Associated with Cancer, PLoS One, 11(5), which are incorporated by reference. In some embodiments, one or more of the primers may be fluorogenic primers, for example, such as a light emitting primer sold under the trade name LUX by Thermo Fisher.

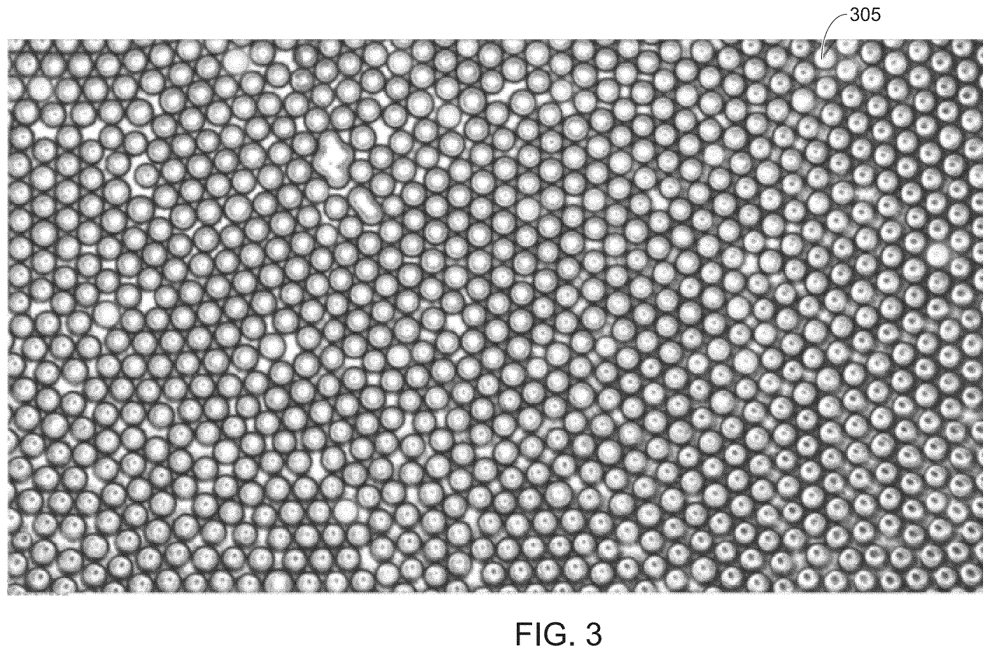

FIG. 3 shows a fluorescent image of dPCR-amplified cfDNA inside pre-templated partitions. The darker (different shading/illuminated in original) droplets 305 are those emitting a signal from a fluorescent reporter, i.e., those including amplicons of cfDNA for subsequent analysis.

In some embodiments, the droplets may be sorted by flow cytometry analysis (FACS) before subsequent analysis (e.g., qPCR). FACS is a process by which a sample mixture of labeled analyte (partitions) is sorted according to their light scattering and fluorescence characteristics into two or more containers. By using FACS, methods of the invention can enrich for partitions that include target amplicons to reduce expenses associated with processing non-target material.

A feature of certain methods as described herein is the use of a polymerase chain reaction (PCR)-based assay to detect the presence of certain oligonucleotides and/or genes of interest in a sample. Exemplary target nucleic acids include those associated with genetic mutations or diseases in a subject. Other target nucleic acids include, for example, those associated with a viral or bacterial infection.

The systems and methods of the invention include using pre-templated instant partitions for pre-amplification of target nucleic acid fragments and subsequently assessing those amplified targets by PCR for cancer mutations. Detection of the cancer mutations can be performed by any number of different PCR based approaches, such as, quantitative PCR (qPCR), quantitative fluorescent PCR (QF-PCR), multiplex fluorescent PCR (MF-PCR), digital PCR (dPCR), PCR-RFLP/real time-PCR-RFLP, hot start PCR, nested PCR, in situ polony PCR, in situ rolling circle amplification (RCA), bridge PCR, picotiter PCR.

In such assays, one or more primers may be sensitive to a cancer mutation. For example, the primers may comprise sequences specific to a particular, non-mutated target such that they will only hybridize and initiate PCR when non-mutated target nucleic acid is present. If the target of interest is present and the primer is a match, many copies of the target may be created using PCR amplification. The presence of target copies may indicate that the target nucleic acid does not have a mutation. To determine whether a particular target amplicon is present in a reaction vessel for mutation detection, the pre-amplified target amplicon may be detected through an assay. For example, in some instances, target amplicon may be detected by probing liquid of the partitions for fluorescence. For example, pre-amplification of target nucleic acid may occur by droplet PCR in the presence of one or more fluorophores that cause target amplicons to fluoresce upon excitement by a specific wavelength. Accordingly, the presence of target amplicons inside a reaction vessel for mutation detection can be confirmed by the presence of a fluorescence signal.

PCR- and real-time PCR-based detection methodologies have greatly improved the analysis of nucleic acids from both throughput and quantitative perspectives. Traditional PCR-based detection assays generally rely on end-point, and sometimes semi-quantitative, analysis of amplified DNA targets via agarose gel electrophoresis; real-time PCR (or qPCR) methods are most often used to quantify exponential amplification as the reaction progresses. Quantitative PCR reactions are monitored either using a variety of highly sequence specific fluorescent probe technologies, or by using non-specific DNA intercalating fluorogenic dyes.

Some preferred systems and methods of the invention include dPCR. Digital PCR (dPCR) is an alternative quantitation method in which target nucleic acids from a dilute sample are individually isolated droplets using PIP encapsulation. The isolated target nucleic acids are amplified in separate reactions in each droplet. The distribution from background of target DNA molecules among the reactions follows Poisson statistics at the terminal and/or limiting dilutions of target DNA. Generally, at a terminal dilution the vast majority of droplets contain either one or zero target DNA molecules. Ideally, at terminal dilution, the number of PCR positive reactions (PCR(+)) equals the number of template molecules originally present. At a limiting dilution, partitions include zero, one, and often more than one target nucleic acid following the Poisson distribution. At the limiting dilution, Poisson statistics are used to uncover the underlying amount of target DNA originally present in a sample.

Methods and systems of the invention can be used to detect and quantify target nucleic acids obtained from a variety of sources. For example, target nucleic acids can be obtained from a solid tissue sample or a fluid sample, such as blood or plasma. Preferably the sample is a fluid sample. Suitable samples may include whole or parts of blood, plasma, cerebrospinal fluid, saliva, sputum, tissue aspirate, microbial culture, uncultured microorganisms, swabs, or any other suitable sample. For example, in some embodiments, a blood sample is obtained (e.g., by phlebotomy) in a clinical setting. Whole blood may be used, or the blood may be spun down to isolate the target nucleic acids.

Preferably, the sample is a blood sample. Obtaining the sample may include performing a blood draw to obtain blood or receiving blood from a clinical facility. In some embodiments, obtaining a sample involves a phlebotomy procedure and collects blood into a blood collection tube such as the blood collection tube sold under the trademark VACUTAINER by BD (Franklin Lakes, N.J.) or a cell-free DNA blood collection tube such as that sold under the trademark CELL-FREE DNA BCT by Streck, Inc. (La Vista, NE). Any suitable collection technique or volume may be employed. A 10 ml sample of blood from a patient infected with a pathogenic microbe may contain only about 1 ng of microbial nucleic acids.

A target nucleic acid may be RNA, DNA, or a mixture thereof. In certain aspects, the methods of the invention include performing reverse transcriptase reaction to produce cDNA of a target RNA. The reverse transcriptase reaction can be performed in the monodisperse droplets. The resulting cDNA can be amplified and detected as described herein. In preferred aspects, the target nucleic acid is a cell-free nucleic acid, which is preferable because it may be taken from blood or plasma via non-invasive procedures.

In certain aspects, methods of the invention include identifying the presence of one or more target nucleic acids in a sample of cfDNA using dPCR before detection of cancer mutations. A dPCR reaction can be used to determine whether a sample is positive for the target nucleic acids. Samples negative for target nucleic acids do not need to be processed. As such, methods of the invention are useful for quickly identifying samples with target nucleic acid for further analysis. This reduces the amount of sample processing performed, thereby reducing material costs.

Methods may include attaching adaptors to amplicons and/or barcoding target fragments to prepare for downstream analysis, for example, sequencing. Any suitable methods may be used to barcode target fragments. The fragments may be barcoded inside droplets. Suitable approaches to attached barcodes to target fragments may include (i) fragmentation and adaptor-ligation (in which adaptors include barcodes); (ii) tagmentation (using transposase enzymes or transpososomes including those sold in kits such as those tagmentation reagent kits sold under the trademark NEXTERA by Illumina, Inc.); and (iii) amplification by, e.g., polymerase chain reaction (PCR) using primers with a hybridization portion complementary to a known or suspected target of interest in a genome and at least one barcode portion that is copied into the amplicons by the PCR reaction. For any of these approaches, the barcodes (e.g., within amplification primers or ligatable adaptors) may be provided free in a solution or bound to a template particle as described herein. In some embodiments, the barcodes are provided as a set (e.g., including thousands of copies of a barcode) in which each barcode is covalently bound to a template particle.

As used herein, barcode generally refers to an oligonucleotide that includes an identifier sequence that can be used to identify sequence reads originating from target nucleic acids that were barcoded as a set with copies of one barcode unique to that set. Barcodes generally include a known number of nucleotides in the identifier sequence between about 2 and about several dozen or more. The oligonucleotides that include the barcodes may include any other of a number of useful sequences including primer segments (e.g., designed to hybridize to a target of interest in a genetic material), universal primer binding sites, restriction sites, sequencing adaptors, sequencing instrument index sequences, others, or combinations thereof. The barcodes may comprise a unique molecular identifier. For example, in some embodiments, barcodes of the disclosure are provided within sequencing adaptors such as within a set of adaptors designed for use with a next generation sequencing (NGS) instrument such as the NGS instrument sold under the trademark HISEQ by Illumina, Inc. Within an NGS adaptor, the barcode may be adjacent to the index portion or the target sequence such that the barcode sequence is found in the index read or the sequence read.

Methods may involve designing PCR primers for amplification of target nucleic acid. A few criteria may be considered when designing a pair of PCR primers. Pairs of primers should have similar melting temperatures since annealing during PCR occurs for both strands simultaneously, and this shared melting temperature is preferably not too much higher or lower than the reaction's annealing temperature. A primer with a Tm (melting temperature) too much higher than the reaction's annealing temperature may mis-hybridize and extend at an incorrect location along the DNA sequence. A Tm significantly lower than the annealing temperature may fail to anneal and extend at all.

Additionally, primer sequences may need to be chosen to uniquely select for a region of DNA, e.g., a region comprising a known cancer mutation, avoiding the possibility of hybridization to a similar sequence nearby. A commonly used method for selecting a primer site is BLAST search, whereby all the possible regions to which a primer may bind can be seen. Both the nucleotide sequence as well as the primer itself can be BLAST searched. The free NCBI tool Primer-BLAST integrates primer design and BLAST search into one application, as do commercial software products such as ePrime and Beacon Designer. Computer simulations of theoretical PCR results (Electronic PCR) may be performed to assist in primer design by giving melting and annealing temperatures, etc.

Selecting a specific region of DNA for primer binding requires some additional considerations. Regions high in mononucleotide and dinucleotide repeats should be avoided, as loop formation can occur and contribute to mis-hybridization. Primers preferably do not easily anneal with other primers in the mixture; this phenomenon can lead to the production of ‘primer dimer’ products contaminating the end solution. Primers should also not anneal strongly to themselves, as internal hairpins and loops could hinder the annealing with the template DNA.

### Examples

Example 1 - uniform PCR amplification by controlled primer concentration with pre-templated instant partitions.

A 10-plex pre-amplification assay was performed in bulk (405) and in pre-templated instant partitions “PIPs” 407. Amplifications were performed with a range of primer concentrations for all targets (0, 1.5, 3.1, 6.3, 12.5, 25, 50 nM). Resulting pre-amplified products were diluted 1:1000 in water and single-plex detection assays for each target were performed by qPCR.

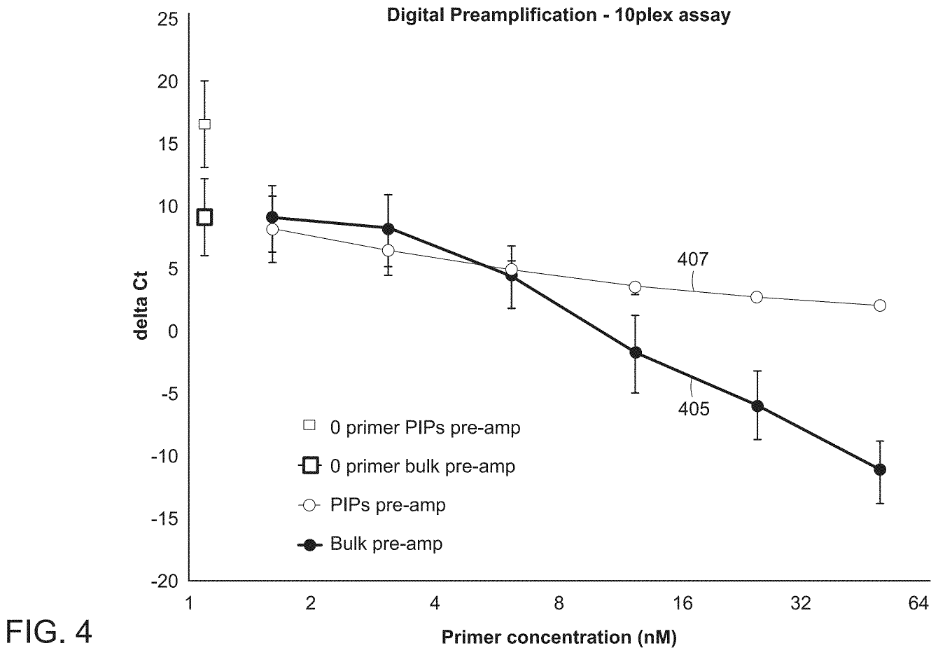

FIG. 4 shows data from a pre-amplification assay. The data are from a 10-plex pre-amplification assay and are plotted as average normalized Ct values across all targets. Error bars are standard deviation across all targets.

PIPs partitioning results in significantly improved amplification uniformity across all targets at all primer concentrations. At primer concentrations below 12.5 nM bulk amplifications are not distinguishable from pre amplification negative controls with no added primers (lower square). In PIPs partitions, samples with low primer concentrations are well separated from no-primer preamplification controls. In the PIPs preamplification, we anticipate that limiting PCR product to available primer concentrations will result in two-fold steps in amplified product in the primer dilution series used. This prediction is confirmed in the digital pre-amplification 407, but not in the bulk amplification 405 results.

### Example 2 —Digital Pre-Amplification and qPCR Detection

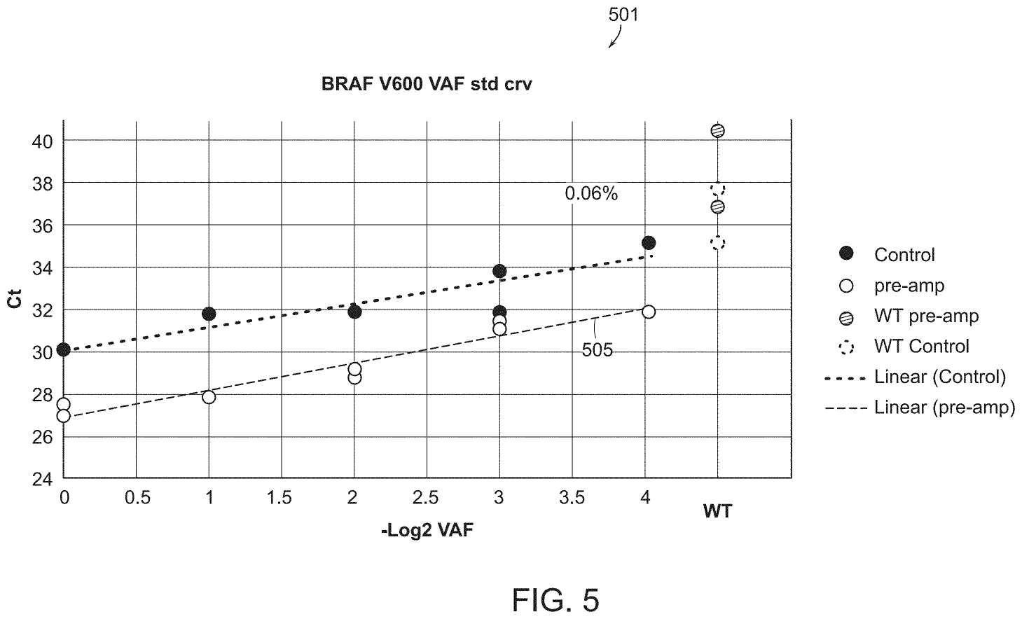

FIG. 5 shows qPCR data comparing amplification efficiency of pre-amplified targets 505 and un-amplified (“Control” targets 507. The data were produced by 23-plex, 15 cycle pre-amplification of targets at 5′ VAF's (1, 0.5, 0.25, 0.13, and 0.06%) and wild type. The pre-amplified products were diluted 200x before detection by allele-specific qPCR.

Allele-specific qPCR of the pre-amplified targets only required 150 ng of DNA for efficient detection of target by qPCR, demonstrating the remarkable ability of pre-amplification to produce high quantities of target nucleic acid. Conversely, without pre-amplification, 1.35 micrograms of total DNA was required for each reaction to provide detectable levels of target nucleic acid by allele-specific qPCR.

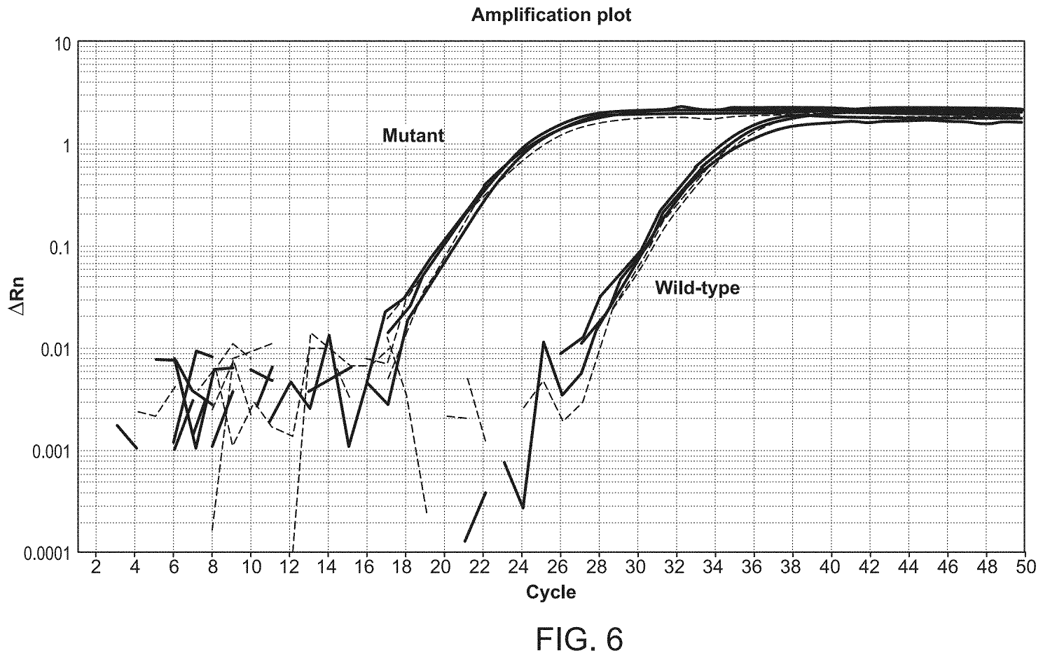

FIG. 6 provides qPCR data that demonstrate the ability of methods of the invention to discriminate between mutant and wild-type nucleic acid. The mutant DNA was pre-amplified as described above by dPCR. 30 nanograms of nucleic acid comprising 1% mutant input was used in the qPCR assay. qPCR was performed with SSP primers.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method for detecting a mutation, the method comprising:
preparing an aqueous solution comprising target nucleic acid and PCR primers; combining the aqueous solution with an oil to create a mixture;
shearing the mixture to form a plurality of partitions, wherein at least a portion of the partitions include a single target nucleic acid and PCR primers;
amplifying the target nucleic acid inside the partitions with the PCR primers to produce a library of amplicons;
splitting the library of amplicons into a plurality of different reaction vessels; and
detecting a mutation from an amplicon in one of the reaction vessels by PCR.

2. The method of claim 1, wherein detecting the mutation is performed with qPCR.

3. The method of claim 1, wherein, during amplification, a number of PCR cycles performed is greater than a number of pairs of PCR primers consumed inside a portion of the partitions.

4. The method of claim 3, wherein a number of unused PCR primers inside the portion of partitions reaches zero before a final PCR cycle is initiated.

5. The method of claim 1, wherein amplifying the target nucleic acid inside the portion of partitions involves at least one PCR cycle comprising zero amplification events.

6. The method of claim 1, wherein the target nucleic acid is uniformly amplified during amplification.

7. The method of claim 1, wherein the target nucleic acid is amplified by digital PCR.

8. The method of claim 1, wherein shearing the mixture comprises using template particles to template the formation of uniformly sized partitions comprising a substantially uniform number of PCR primers.

9. The method of claim 1, wherein the target nucleic acid is a cell free nucleic acid.

10. The method of claim 9, wherein the cell free nucleic acid is pre-identified as a recurrently protected genomic region.

11. The method of claim 9, wherein the cell free nucleic acid is isolated from a urine sample, a blood sample, or a sputum sample.

12. The method of claim 1, wherein the target nucleic acid is approximately 60-90 base pairs in length.

13. The method of claim 1, further comprising calculating a concentration of PCR primers to add to the aqueous solution such that a substantial number PCR primers are exhausted before amplification is complete.

14. The method of claim 2, wherein qPCR is performed with modified primers, the modified primers comprising one or more of a locked nucleic acid primer, a 2-tailed primer, or a light emitting primer.

15. The method of claim 1, wherein at least one of the PCR primers is fluorogenic.

16. The method of claim 1, wherein different ones of the PCR primers comprise sequences complementary to different molecules of target nucleic acid.

17. The method of claim 1, wherein the plurality of different reaction vessels contains reagents for detecting one or more cancer mutations.

18. The method of claim 17, wherein one of the plurality of different reaction vessels comprises a first reagent for detecting a first cancer mutation and a second one of the plurality of different reaction vessels comprises a second reaction for detecting a second cancer mutation.

19. The method of claim 17, wherein the reagents comprise primers for qPCR.

20. The method of claim 19, wherein, in the presence of a cancer mutation, the primers fail to hybridize with amplicons, thereby indicating the presence of mutation.
